(19) United States
(12) Patent Application Publication
(10) Pub. No.: US 2018/0352937 A1
(43) Pub. Date: Dec. 13, 2018

(54) SYSTEM FOR ANALYSIS AND ACTIVATION AND/OR LOCALISED RELEASE FOR THE HUMAN FACE

(71) Applicant: WB Technologies, Paris (FR)

(72) Inventors: Stanislas Vandier, Paris (FR); Elodie Burty-Valin, Villejuif (FR); Benjamin Prunier, Clermont Ferrand (FR); Jean-Roch Meunier, Aulnay-sous-Bois (FR); Jean-François Grollier, Paris (FR); Claude Dubief, Le Chesnay (FR)

(73) Assignee: WB Technologies, Paris (FR)

(21) Appl. No.: 15/757,284

(22) PCT Filed: Sep. 4, 2015
(86) PCT No.: PCT/FR2015/052354

§ 371 Date: Mar. 2, 2018

(51) Int. Cl. A45D 44/00; A61N 1/04; A61M 37/00; A61N 5/06

(52) U.S. Cl. CPC A45D 44/002; A61N 1/0428; A61B 5/01; A61N 5/062; A61M 37/00

(57) ABSTRACT

The invention relates to a system for analysis and activation and/or active and localised release, comprising a mask (1) for applying to at least part of the face of the user, provided with at least one device (2) for analysing the skin of the face and at least one means for the active and localised release (3) of dermocosmetic formulations from at least one storage means.

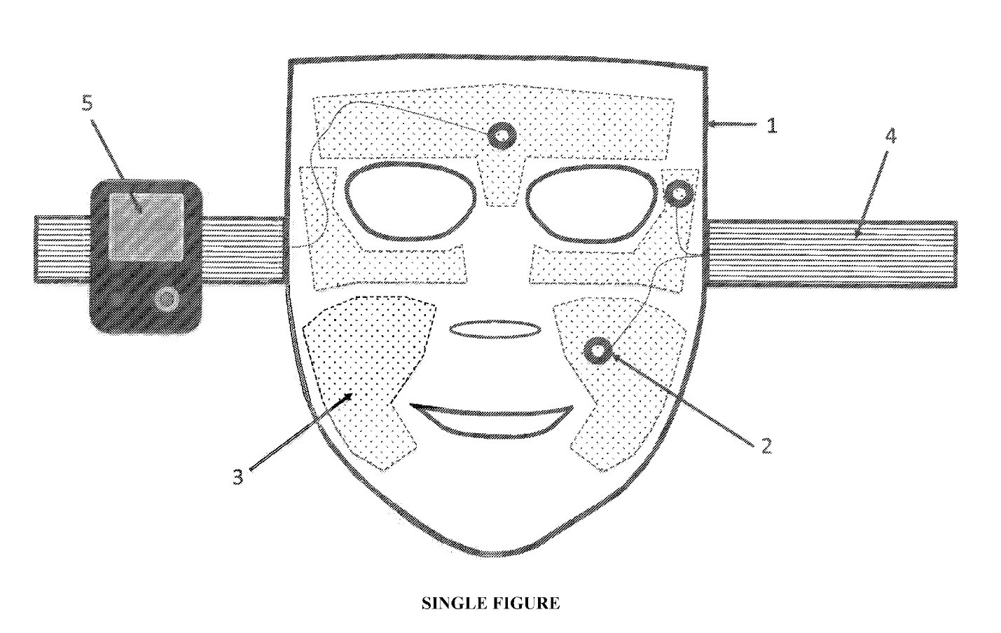

The technical field of the invention is that of measuring devices and more particularly devices for measuring physico-chemical and biological parameters of the skin.

The measurement of variations in the cutaneous parameters of various zones of the face and/or body from day-to-day and at home is not currently possible.

However, both women and men need to better assess the actual state of their skin with scientific and objective criteria, in order to better care for it, preserve its health and its beauty, and to apply the right products at the right moment on the basis of objective criteria.

Personalized monitoring over time is paramount since cutaneous parameters are influenced by a number of criteria such as: age, gender, ethnic origin, the environment and circadian cycles, which by definition are specific to each individual and to lifestyle.

The skin, in particular that of the face, is heterogeneous and consists of a plurality of zones having characteristics that are very different in terms of hydration, temperature, rate of production of sebum, barrier function, etc. Specifically, scientific studies have shown that the forehead, cheeks, nose and chin have particular, different and specific properties (Lopez et al., 2000, Skin Research and Technology; Wa and Maibach, 2010, Skin Research and Technology; Song et al., 2015, International Journal of Cosmetic Science).

The skin is not a uniform space but an organ combining at least four forms of heterogeneity:

A heterogeneity from one individual to the next,

For each individual, a spatial heterogeneity depending on the zone of the body or even of the face itself,

A temporal heterogeneity because of variation during the day and over an individual's life,

A cellular and molecular heterogeneity related to the various compartments that compose it (epidermis, dermis, hypodermis).

Regular monitoring and a capacity to better evaluate the ways in which the skin has changed during the day allow dermo-cosmetic routines to be personalized and new solutions to be implemented that are more effective because they are targeted and take into account the effects of the environment encountered and the biological mechanisms behind the response of the skin to the exogenous compounds applied.

For all these reasons, there is a need for a system allowing such information to be gathered in order to allow dermo-cosmetic analyses to be carried out and advice to be given to consumers. Specifically, knowledge of the specific characteristics of each zone of the face would make it possible to make an overall and local assessment of the state of the skin, to recommend the application of dermo-cosmetic products and to determine and/or compare the effectiveness of dermo-cosmetic products in a way that is more precise, more personalized and therefore more optimal in terms of the effectiveness of the results.

Regarding the prior art, mention may be made of documents KR20120063169, CN203663253 and US2014268099. However none of these documents combines monitoring/tracking of the state of the skin over time, personalized advice incorporating environmental and behavioral characteristics and a controlled treatment on demand of specific zones depending on these recommendations.

One subject of the invention is a system for analyzing and actively and locally releasing and/or activating, comprising a mask intended to be applied to at least one portion of the face of the user equipped with at least one device for analyzing the skin of the face and with at least one means for actively and locally releasing and/or activating dermo-cosmetic formulations originating from at least one storing means.

The mask may comprise a thermoformable polymer and fibers.

The device for analyzing the skin of the face may comprise at least one specific sensor able to analyze at least one predefined zone of the face.

A specific sensor may be chosen from temperature sensors, bio-impedance sensors, sensors sensitive to a concentration of at least one ionic species, biosensors, means for generating and/or sensors of optical stresses, sensors for measuring the environment.

The predefined zones of the face may comprise the forehead, the cheekbones, the nose, the chin, the L-shaped zones containing the areas just below the eyes and the crow's feet or the zone of the cheeks.

The means for actively and locally releasing dermo-cosmetic formulae may be able to control the flow of ingredients contained in at least one storing means toward at least one predefined zone of the face, depending on the measurement results of at least one device for analyzing the skin, by contracting electrical, thermal, mechanical, etc. actuating means that make contact with at least one storing means.

The storing means may be comprised in or on the system or placed on at least one predefined zone of the face before the mask is applied.

The locally activating means is able to create local conditions that are liable to increase the effectiveness of the dermo-cosmetic formulae locally released onto or applied beforehand to the skin.

To create local conditions liable to increase the effectiveness of the chemical compounds, the locally activating means may comprise an electrical heating system, a Peltier system, or a system of infrared diodes able to control the temperature between the mask and the skin.

To create the local conditions liable to increase the effectiveness of the dermo-cosmetic formulae, the locally activating means may comprise light-emitting diodes able to produce a continuous or pulsed irradiation of the skin.

To create local conditions liable to increase the effectiveness of the dermo-cosmetic formulae, the locally activating means may comprise electrophoresis electrodes and/or cutaneous electrophysiological electrodes.

The system for analyzing and locally releasing and/or activating has the advantage of being able to alert the user to factors aggravating undesirable cutaneous changes (ageing, irritation, dehydration, acne, etc.) and of being able to optimize the logic of routines for caring for the skin of the face (cleaning and care),

The system for analyzing and locally releasing and/or activating also has the advantage of treating the face (or zones of the face) on demand and depending on recommendations and taken measurements, and of making available to professionals (spas, institutes, dermo-cosmetics manufacturers) a new tool allowing data to be harvested from extensive geographical zones (in particular polluted zones) in order to allow new and more effective cosmetic formulae to be developed.

Other aims, features and advantages of the invention will become apparent on reading the following description, which is given solely by way of nonlimiting example with reference to the appended drawing, in which the single figure illustrates the system according to the invention.

The SINGLE FIGURE shows a system for analyzing and locally releasing and/or activating, comprising at least one device 2 for analyzing the skin of the face and at least one means 3 for locally releasing dermo-cosmetic formulae originating from at least one storing means, these means and device being placed in a mask 1 intended to be applied to all or some of the face of the user. The mask 1 is made from: a thermoformable polymer (for example based on polycaprolactone as described in the patent WO2014/170220A2 of the company TOMTEC), or any other polymer alone or in a blend; and natural, artificial or synthetic fibers that are nonwoven, spun, woven or knitted, pure or mixed, such as (non-exhaustive list) cotton fibers, linen fibers, bamboo fibers, viscose fibers, polyamide fibers, polyester fibers, silicone fibers, polyolefin fibers (such as fibers of polyethylene and polypropylene), and mixtures of fibers of different natures. The thermoformable polymer and the fibers are combined either by flowing the polymer over the fibers, or subsequently by adhesively bonding the fibers to the polymer sheet or by any other method allowing the fibers to be superposed on the delivered sheet of polymer.

A device 2 for analyzing the skin of the face comprises at least one specific sensor, at least one bioimpedance sensor (for example ST microelectronics, HM301D) and proprietary wet or dry electrodes in order to measure the hydration of the surface layers of the epidermis, at least one sensor sensitive to a concentration of at least one ionic species, in particular a sensor of pH with wet or dry electrodes (for example Brongersma et al, 2014, Phys. Status Solidi C), of oxidant stress via the detection of oxidized molecules (proteins, lipids, carbohydrates, etc.) and/or of antioxidant species such as vitamins A and C, glutathione, etc., of pollution (PM 10 and/or PM 2.5 particulates, pollen, microbiological organisms, heavy metals such as zinc (Zn), copper (Cu), nickel (Ni), lead (Pb), chromium (Cr), selenium (Se), arsenic (As), mercury (Hg) and cadmium (Cd)), of temperature (for example Cantherm, MF51E thermistors) allowing cutaneous temperature to be measured or at least one sensor for detecting biochemical species, oxidation effects and other molecules of interest on the surface of the skin (in particular sebum, microbiota, metabolites, desquamated cells, vitamins, growth factors, etc.) at least one means for generating and/or sensor of optical stresses allowing biological, biochemical or chemical species on the surface of the skin to be imaged (with or without magnification), irradiated (UV, IR, visible, fluorescence, blue light, etc.), analyzed and/or located with or without direct contact with the surface of the skin (http://www.microphonelens.com; http://www.rambus.com), at least one fiber allowing information (temperature, conductivity, pH, etc.) to be captured over its entire length or at points of intersection of a mesh (Quandt et al., 2015, Adv Healthc Mater), at least one environmental sensor such as for measuring temperature, humidity (e.g.: Silicon Labs, Si7020), pressure (e.g.: Bosch, BMP180), distortion or “slipperiness” via measurement of the scrub resistance of a material using suitable pressure and force sensors (Nakatani et al, Skin Res Technol. 2013 Feb; 19(1):pages 332-338 and for example all the communicating sensors of the company Strenchsense™)

Biochemical species, oxidation effects and other molecules of interest may preferably be detected on the surface of the skin using electrochemical electrodes that are connected to systems for measuring electrical impedance (electrical impedance spectroscopy) (Eom et al, 2011 Phys. Rep.-Rev.Sec. Phys. Lett. 503 115-163) or microcantilever systems conjugated with polymers or antibodies (LumiMEMS™ from Panorama Synergy).

The sensors for monitoring/tracking the state of the skin may be discrete (point sensors) and interconnected by technologies for conducting electrical energy and information, these technologies being compatible in particular with a textile carrier (such as those developed by Cityzen Sciences and the consortium Smart Sensing) or indeed being printed with ad hoc conductive inks on flexible polymeric, silicone or textile carriers. These sensors may also take the form of interconnectable, weavable optical fibers allowing measurement over the length of the fiber and not only at a single point.

The sensors are connected by flexible wires, which are borne by pliant electronic flexes or printed with conductive inks that may be integrated into elastic bands or straps 4 for maintaining the mask 1 or positioned directly on the motherboard. This connecting system may also form the system for connecting to the data-transferring or recharging system.

An analyzing device 2 is able to analyze a predefined zone of the face, for example the forehead, the cheekbones, the nose, the chin, the L-shaped zone containing the area just below an eye and the crow's feet or the zone of the cheeks. This system for analyzing and locally releasing comprises a plurality of devices allowing the various zones of the face to be analyzed. Alternatively, an analyzing device 2 comprises a plurality of sensors able to analyze a plurality of zones of the face simultaneously and/or by cross-analysis.

The interior of the mask 1 includes, on the entirety of its surface or in predefined zones, at least one means 3 for locally releasing dermo-cosmetic formulae.

A means 3 for locally and/or controllably releasing dermo-cosmetic formulae allows the flow, diffusion or migration of dermo-cosmetic agents and ingredients contained in at least one storing means (taking the form of patches, hydrogels, fibers, etc.) toward at least one predefined zone of the face to be controlled, for example via a mechanical, electrical, biochemical or thermal stimulation that depends on the measurement results of at least one device 2 for analyzing the skin.

An example of such a locally releasing means 3 comprises mechanical actuating means able to generate a contraction in reaction to an electrical pulse. Such means may be a piezoelectric polymer grafted onto textile fibers (artificial-muscle technology used in robotics and commercialized by PIEZOTECH, subsidiary of Arkema for example) and/or a film of polymers possessing the same properties. The application of an electrical current then allows the carrier to be “contracted” on demand, thus allowing a mechanical stress to be produced that is used to break capsules containing the agent when the latter is encapsulated.

Such mechanical actuating means are combined with means for storing dermo-cosmetic formulae of the type in which the formulae are encapsulated in coating systems or capsules that are sensitive to friction, temperature, pH and/or mechanical actions.

The mechanically actuating means are combined with the means for storing by imbibing, impregnating or direct spraying onto the mechanical actuating means.

Alternatively, at least one portion of the storing means consists of a removable patch (hydrogels or fibers for example) placed on the predefined zones of the face prior to the use of the system for analyzing and actively and locally releasing. The reversible and controlled contraction of the fibers causes the capsules or devices containing the demo-cosmetic agents and ingredients to burst and said agents and ingredients to be freed, on demand.

The locally activating means 3 may also be able to create local conditions liable to increase the effectiveness of the dermo-cosmetic formulae released locally or applied beforehand to the skin. The local conditions may comprise a modified temperature, and exposure to a (pulsed, IR, UV, fluorescent, blue light, etc.) light source. In such an embodiment, the agents may be encapsulated in capsules that are sensitive to the radiation in question, then causing them to be released.

In one particular embodiment, the locally activating means 3 comprises systems for regulating the temperature of the skin either over the entirety of the area covered by the system for analyzing and locally releasing, or in one of the predefined zones. This temperature regulation allows temperatures comprised between 37 and 45° C. to be achieved controllably or the surface of the skin to be cooled to temperatures ranging for example from 5° C. to 25° C. (or 5° C. below room temperature) for a length of time that is modulatable by the user via a mobile application.

The temperature is regulated via a closed feedback loop depending on the temperature measured by the temperature sensors associated with the affected predefined zones.

The temperature will possibly be controlled by various systems such as an electrical heating system, a Peltier system, or a system of infrared diodes.

The electrical heating system employs joule heating to heat the surrounding medium. It comprises one or two wires, of variable diameters, of the “magnet wire” type, allowing information and electrical signals to be conveyed from set zones of the mask (carrier and Gateway) to detachable and washable zones.

The Peltier system makes it possible to induce an increase or decrease in the temperature of the internal face of the mask 1, in contact with the skin, and an opposite modification of the temperature of the external face of the mask 1.

The system of infrared diodes allows heating via local IR irradiation. The heat generated may be dissipated/spread beyond the diode or Peltier system by a metal plate or polymers or fibers of suitable thermal conductivity.

In another embodiment, the locally activating means 3 comprises electrophoresis and/or cutaneous electrophysiological electrodes combined with storing means taking the form of hydrogel patches that are, for example, placed on the skin prior to use of the system for analyzing and locally actively releasing. The micro-electrophoresis (2.5 to 12 V maximum) resulting from the use of these electrodes allows migration, within the hydrogel patches, of dermo-cosmetic agents and ingredients contained in these patches to be achieved, thus making them bioavailable for the skin, i.e. accessible to the upper layers of the epidermis for absorption. Alternatively, these electrodes allow cutaneous micro-currents liable to increase the effectiveness of dermo-cosmetic formulae applied to the superficial surface layers of the epidermis before the mask 1 is donned to be created (see for example Feeligreen).

In another embodiment, the locally activating means 3 comprises diodes (for example emitting: IR, UV, fluorescent light or blue light) able to produce a pulsed or continuous irradiation of the skin over the entire area of the system for analyzing and actively and, locally releasing and/or activating or in at least one of the predefined zones.

The irradiation may be carried out at different wavelengths depending on the results of the analysis and on the state of the skin. For example, an irradiation with red light and/or infrared is employed for zones containing acne and/or rosacea in particular for its anti-inflammatory effects and the effectiveness with which it decreases the production of sebum. An irradiation with blue light is employed to stimulate cutaneous scarring and to decrease bacterial activity.

To this end, all or some of the system for analyzing and locally and actively releasing and/or activating comprises at least one light-emitting diode and preferably 2 to 12 diodes per preset zone. The illumination is continuous over a defined period (e.g.: 1 minute) or pulsed via the use of turn-on/turn-off cycles of preset frequencies and may be combined with the various available irradiation wavelengths. Alternatively, the means 3 for controllably and locally releasing and/or activating may be triggered on demand, optionally from the mobile application. The smart phone, tablet or computer may be able act as a remote display and display operating data of the system (environmental and physiological data, advice and recommendations, user remarks).

The system for analyzing and actively and locally releasing and/or activating moreover comprises: at least one rigid or flexible rechargeable battery (for example solutions sold by the company EMG2); a system for storing data and a system for transmitting data, which are connected together via a motherboard (microprocessor, passive components positioned on a PCB that is printed on a flexible carrier, etched using chemical processes, or printed using a 3-D process); an analog/digital interface; a system for controlling and recharging the battery; a system for acquiring data; a system for storing data locally (micro-SD card); a communication system for transferring data, this system possibly being a wired system, a Bluetooth system, a Wi-Fi system, a radio system, etc.; and a measurement control system of the “one button/one measurement” type. These various systems may be integrated into a removable, appended housing 5 that is optionally flexible, rigid or semi-rigid, miniaturized and, depending on the version brought to market, waterproof or non-waterproof.

The method for analyzing and locally and actively releasing and/or activating comprises innovative algorithms allowing suitable dermo-cosmetic formulae and beauty routines to be recommended depending on the analysis of the data generated by the device 2 for analyzing the skin.

The method for analyzing and locally and actively releasing and/or activating moreover comprises a mobile application in order to allow data and personalized advice to be displayed, an online sales site to be accessed, etc.

The analysis of the data transmitted by the sensors is carried out initially by virtue of a mobile application (smart phone, tablet, etc.) containing algorithms for crossing over environmental data and cutaneous measurements discretely in the morning and/or evening or at any other opportune moment of the day. The application also allows data to be displayed and the user to get advice on the state of his or her skin, recommendations as regards prevention, warnings, recommendations as to products to use with respect to his or her dermo-cosmetic problems (via combination of environmental data and physiological data of the skin), beauty routines to implement, etc.

The data may be stored locally on the smart phone or tablet or transmitted via a 3G or Wi-Fi connection to a database and to a server allowing synthetic data relating to the hydration state of the skin of the user to be exchanged initially. The user in return receives his or her analyzed data and may view them via his or her mobile application.

The above allows an analysis to be carried out, in real-time, using the procedure described below:

1) real-time information from the sensors is stored then normalized depending on reference thresholds of each of the sensors; (standard deviation, moving average)

2) the data are then grouped and analyzed statistically in order to compare them to models; (trend estimation)

3) the values output from the models are transferred to the mobile application; (secure transfer of BLE4.0 type)

4) the data are then reworked and placed in relationship with all the other data acquired beforehand in order to display normalized indicators of the state of the skin via the mobile application (indicators grouped depending on seasonality, ACP, linear regression, weighting and a decision workflow into three categories: environmental parameters, lifestyle, physiological parameters)

5) error-detecting algorithms will be present in order to analyze in real-time the potential deltas of the sensors.

Another subject of the invention is a method for cosmetically treating the skin, characterized in that at least one system for analyzing and actively and locally releasing and/or activating such as defined above is applied to at least one portion of the face of the user, and in that, after one or more zones of the skin of the face have been analyzed by the analyzing means of the system, a suitable dermo-cosmetic formula is actively and locally released and/or locally activated in order to create local conditions liable to increase the effectiveness of the dermo-cosmetic formulae released locally onto or applied beforehand to the skin.

1. A system for analyzing and locally and actively releasing and/or activating, characterized in that it comprises a mask intended to be applied to at least one portion of the face of the user equipped with at least one device for analyzing the skin of the face and at least one means for actively and locally releasing and/or activating demo-cosmetic formulations originating from at least one storing means.

2. The system as claimed in claim 1, wherein the mask comprises a thermoformable polymer.

3. The system as claimed in claim 1, wherein the device for analyzing the skin of the face comprises at least one specific sensor able to analyze at least one predefined zone of the face.

4. The system as claimed in claim 3, wherein a specific sensor is chosen from temperature sensors, bio-impedance sensors, sensors sensitive to a concentration of at least one ionic species, biosensors, means for generating and/or sensors of optical stresses, sensors for measuring the environment.

5. The system as claimed in claim 3, wherein the predefined zones of the face comprise the forehead, the cheekbones, the nose, the chin, the L-shaped zones containing the areas just below the eyes and the crow's feet or the zone of the cheeks.

6. The system as claimed in claim 1, wherein the means for actively and locally releasing dermo-cosmetic formulations is able to control the flow, the migration or the diffusion of dermo-cosmetic agents and/or ingredients contained in at least one storing means toward at least one predefined zone of the face, depending on the measurement results of at least one device for analyzing the skin, by contracting electrical, thermal or mechanical actuating means that make contact with at least one storing means.

7. The system as claimed in claim 1, wherein the storing means are comprised in the system or placed on at least one predefined zone of the face before the mask is applied.

8. The system as claimed in claim 1, wherein the locally activating means is able to create local conditions that are liable to increase the effectiveness of the dermo-cosmetic formulae locally released onto or applied beforehand to the skin.

9. The system as claimed in claim 8, wherein, to create local conditions liable to increase the effectiveness of the dermo-cosmetic formulae, the locally activating means comprises an electrical heating system, a Peltier system or a system of infrared diodes able to control the temperature between the mask and the skin.

10. The system as claimed in claim 8, wherein, to create the local conditions liable to increase the effectiveness of the dermo-cosmetic formulae, the locally activating means comprises light-emitting diodes able to produce a continuous or pulsed irradiation of the skin.

11. The system as claimed in claim 8, wherein, to create local conditions liable to increase the effectiveness of the dermo-cosmetic formulae, the locally activating means comprises electrophoresis electrodes and/or cutaneous electrophysiological electrodes.

12. A method for cosmetically treating the skin, characterized in that at least one system for analyzing and actively and locally releasing and/or activating according to claim 1 is applied to at least one portion of the face of the user, and in that, after one or more zones of the skin of the face have been analyzed by the analyzing means of the system, a suitable dermo-cosmetic formula is actively and locally released and/or locally activated in order to create local conditions liable to increase the effectiveness of the dermo-cosmetic formulae released locally onto or applied beforehand to the skin.
